(19) United States
(12) Patent Application Publication
(10) Pub. No.: US 2023/0086712 A1
(43) Pub. Date: Mar. 23, 2023

(54) Two-Sided Digitized Healthcare Assets Platform

(71) Applicant: Dov Biran, Scarsdale, NY (US)

(72) Inventor: Dov Biran, Scarsdale, NY (US)

(21) Appl. No.: 17/478,927

(22) Filed: Sep. 19, 2021

(51) Int. Cl. G16H 40/20; G16H 20/10; G16H 10/20

(52) U.S. Cl. CPC G16H 40/20; G16H 20/10; G16H 10/20

(57) ABSTRACT

A two sided digital platform for medical patients and medical providers is described in this document. On the provider side, users are able to create digital healthcare assets and programs which prescribe a set of instructions to patients. These instructions may vary from a detailed long-term medical care-plan to a quick survey of questions to be filled. From the patient side, users may browse medical programs which are made open by providers, as well as be assigned personalized programs by their caregivers. Feedback is given to the system by both providers and patients, which feeds a recommendation mechanism that suggests patients relevant programs that may help them achieve healthcare and lifestyle goals. Users are able to view their programs in a compartmentalized view, whether it is providers editing their programs under both public and private settings or patients reviewing all of the programs they are enrolled in and assigned divided based on the programs' inherent instructions. Patients' adherence to programs is constantly reviewed, and intelligent notifications and motivations are presented to patients to ensure constant improvement and optimal progress.

### TECHNICAL FIELD

The invention relates to the medical information field as well as dual-sided technological platforms. Specifically, the invention relates to the digital healthcare field, and the interfaces between patients and providers, both remote and in-person.

### BACKGROUND ART

Digital technology and telemedicine have expanded modern healthcare to include a large number of ways for patients to be seen and reviewed by professionals in a remote and distant manner. Many traditional doctor's visits now take place online, and patients have a much larger role in their own healthcare, as physical inspection and proximity are no longer required for many medical treatments and procedures. Progress in medicine and healthcare allows patients to spend most of their care and recovery time from home.

These advancements have allowed providers to give care to a greater number of people, and for patients to seek care and advice on many health aspects from many medical sources. With this wide distribution of medical care and knowledge spreading across many medical fields and countless patients and providers world-wide, managing such a large network of information in a way that still allows for seamless interactions between patients and providers would work for the benefit of both sides.

As patients seeking care and advice go through traditional means of exploration, calling and scanning the internet for care options and programs that fit their needs, a singular source of such professional resources where providers can list medical care plans and advice would reduce time spent searching for care and increase time spent improving, recovering and working towards one's health goals. Simultaneously, such a system would give providers a tool to directly monitor patients' progress and even assign patients with personalized medical plans and instructions.

### SUMMARY

A two sided platform which allows medical caregivers to create, assign to patients, and view others' medical care plans and programs (also referred to as digital assets) is described in this document. Caregivers such as doctors and nurses may use the system to concoct a variety of plans, from medical questionnaires to detailed medical recovery and medication prescription plans. Accessibility specifications would be made for each such plan, as providers could decide who may view and enroll in their designed programs. After assigning plans to their specific patients, either between visits of regular checkups or following a final discharge, providers can view and track their patients' progress, and monitor the statuses of enrolled individuals.

The platform would provide patients with direct access to any provider's listed plans and programs, giving them the capabilities to browse through guidance and advice on a variety of topics and health issues. Patients could download or enroll in openly listed programs and track their own progress as they work towards their goals and objectives. After completion, patients' results and feedback would be stored and shown to relevant providers, giving them the opportunity to analyze their programs' efficacy and their patients' performances.

The system would also take these results and analyze them on its own. A process of intelligent recommendation would be driven by this data, as patients with similar characteristics and goals could inform the system of potential programs that match patients' objectives and interests, and align the patients with plans and programs that have direct relevance and proven efficacy to help individuals achieve certain goals. This would make the process of searching for care and improving patients' health even more seamless and swift.

Additionally, the system will employ a feedback and motivation aspect to ensure patients remain on track to complete the plans and programs they are enrolled in. An intelligent sub-system would ensure progress is maintained and goals are achieved by directly addressing and concentrating notifications on patients who do not adhere to assigned plans.

### DESCRIPTION OF DRAWINGS

FIG. 1 is an illustration of a visual interface displaying the patient a comprehensive view of their active programs and plans.

FIG. 2 is an illustration of the interface allowing providers to create plans and programs.

FIG. 3 is an illustration of the program-building interface where providers create and specify the scope of a program to be published.

FIG. 4 is an illustration of the interface allowing providers to specify where a program is to be published to.

FIG. 5 is an illustration of an interface displaying the schedule of medications assigned to a patient with clear instructions of application.

FIG. 6 is a diagram of the data repositories and software modules required to store relevant information for the system and execute necessary processes.

FIG. 7 is a diagram showing the possible digital assets that users may create and obtain on the platform.

FIG. 8 is a flow diagram illustrating the real-time alerting and dynamic response capabilities of questionnaires and surveys created on the system.

### DETAILED DESCRIPTION

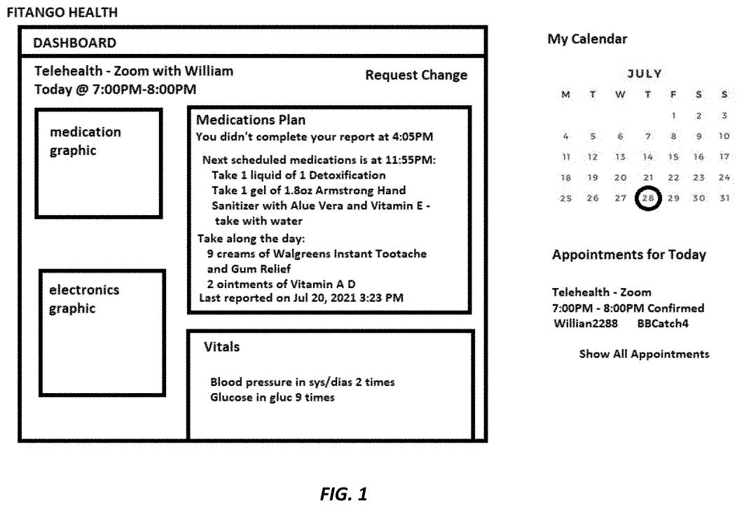

FIG. 1 shows the system as it would be seen to a patient reviewing all of their currently enrolled plans. Referred to as the ‘dashboard’, this interface lists the patient's plans and programs in the center of the interface, along with any upcoming meetings or visits. The listed plans, which in the illustration are a medications plan and a vitals plan, are detailed with the patient's adherence to the plan, required steps and instructions that correspond to the patient's progress in the plan, and the last time the patient reported on the program. In the illustration, an example medication plan shows the doses required for each of the patient's assigned medications along with a warning that the patient has not adhered to the program recently. On the right hand side of the illustration, a schedule is presented to the patient with upcoming meetings and visitations.

This interface is an example of the implementation of the system where users may view their chosen and enrolled programs outside of the two-sided platform. As patients select programs which help them achieve goals and arrive at improved medical statuses, this dashboard would give each patient a private view on their progress and improvement. The interface will also help patients adhere to their plans by alerting them on missed progress and any upcoming professional visits and meetings.

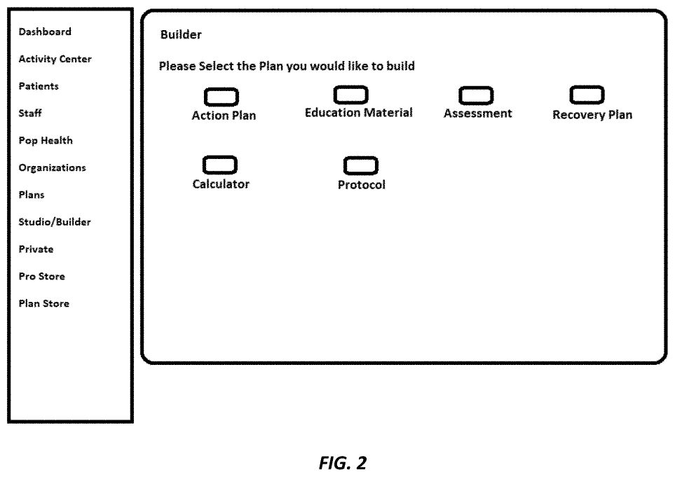

FIG. 2 illustrates a digital selection board available to providers when creating a program to be published on the platform. This ‘Studio/Builder’ presents providers with templates of programs, such as ‘Action Plans’ and ‘Assessments’ seen in the figure. Each program has a unique specification and requires different inputs from patients while providing them with different outputs. For example, an assessment plan may simply ask patients to digitally answer preset questions, while an action or recovery plan could require patients to perform physical actions outside of the platform to progress toward a certain medical goal.

FIG. 3 shows the plan building interface for a specific program type. In this figure, the window exhibits how a provider may build the body of an action plan to be published on the platform. Such an action plan may require patients to perform any of a multitude of actions. On the right hand side, the list of potential inputs represents these actions. The provider may specify the plan to require patients to input a confirmation of a certain condition, or a medical tracker for the patient to relay to the system. The right hand side panel also gives the provider a selection of outputs to be given to the patient as they progress through the program. Such outputs may be a simple text or potentially a video to help the patients with the program. In the middle panel, providers may select time windows by which progress on the program needs to be done, and other fields to be shown to the enrolled patient. The left-hand side panel allows providers to add more sections to the program, which give several layers of inputs and outputs to the plan. Patients could be prompted with all layers at once, or progress to each layer as its predecessor is completed.

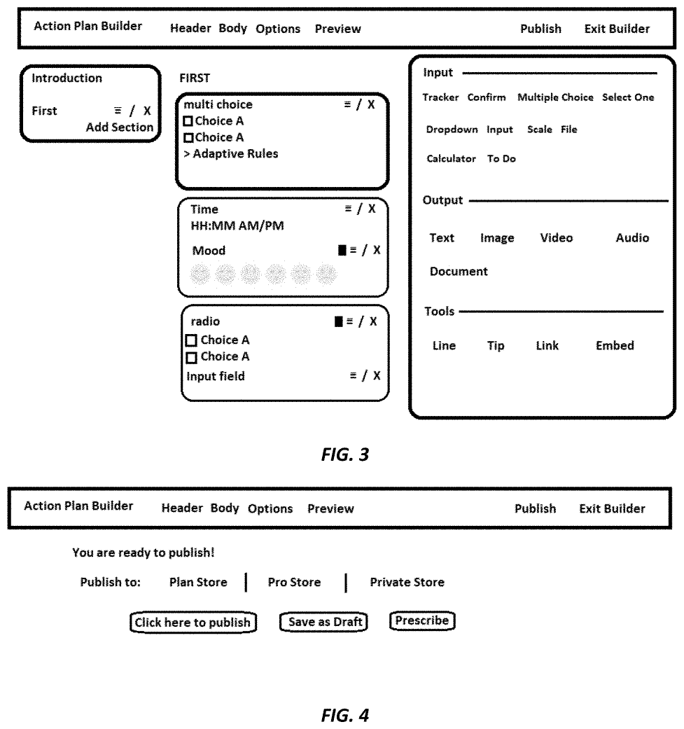

FIG. 4 illustrates the ultimate panel in the plan-building interface. Once the plan is built, a provider would select where to publish the plan to. Different plan stores may have different inherent accessibility rights. For example, a provider may select to publish a program to all patients on the platform, referred to in the figure as the “Plan Store”. Alternatively, the provider may publish the digital asset only to be seen by other providers, to an option referred to in the figure as the “Pro Store”. Each publication choice would specify this destination. Another option for the providers is to save a program as a draft or completed but only to be accessible to them, and not allow any other user of the system to view or interact with it, referred to the figure as the “Private Store” option. Once the accessibility decision is made, a provider may save the plan under the corresponding storage and publish it accordingly.

FIG. 5 presents an implementation of an interface embodying a specific plan type. Other than a singular dashboard as presented in FIG. 1, the system may provide users with interfaces to interact with their programs in a more granular fashion. In FIG. 5, a patient's medication-specific program is displayed, listing all of the medications assigned and divided into categories. This division could represent any segmentation of the medications, and the illustration shows a dosis-based compartmentalization. The top-most category is populated with medications that need to be taken at specific times. Along with this category, the interface presents a detailed schedule of times during which action is required from the patient and the specific amount of medication to be taken. Other categories in this segmentation are populated with medications that do not fit into a specific schedule of application. Nonetheless, the interface still provides the patient with relevant information pertaining to the frequency and quantity of the medications' consumption.

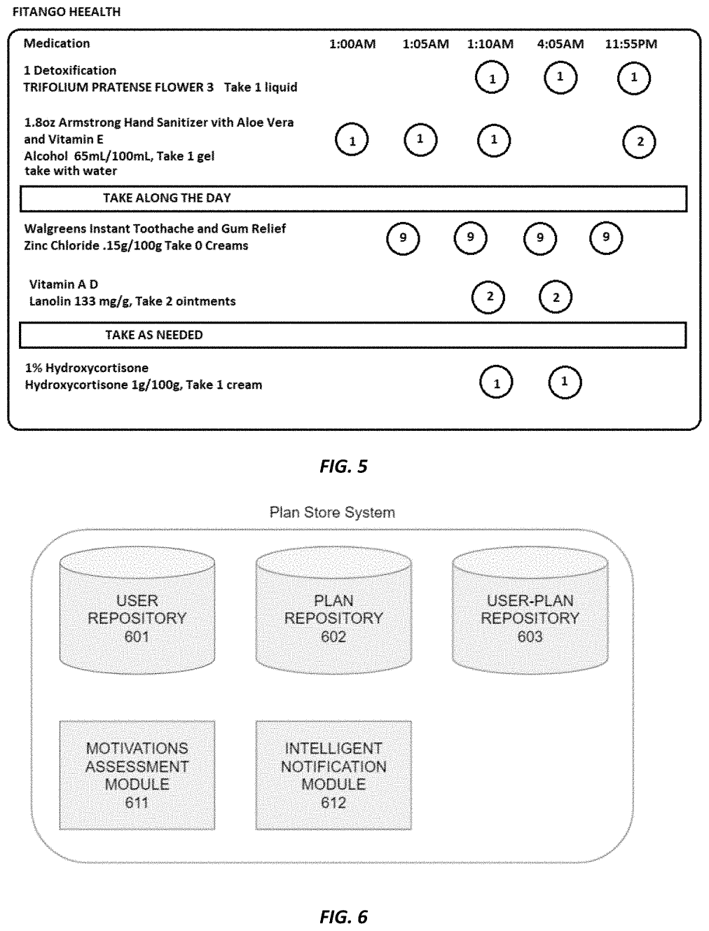

FIG. 6 exhibits the technical components that comprise the invention and system that embodies it. One data storage component, the user repository 601, stores information regarding the patients using the system. General identification information along with medical and demographic data can be stored in such a repository. A second storage component, the plan repository 602, similarly stores information relevant to all plans and programs templates created on the system. Whether they are stored in a provider's private storage or are public for all users to see, the information regarding all programs and plans is maintained in this repository. The last storage component, the user-plan repository 603, combines information from the two other repositories and specifies the permissions users have to any plan. Information regarding assignment, enrollment, and progress made by a patient is stored in this repository. The motivation assessment module 611 represents the processes and programs that determine how likely a new patient is to adhere to plans they are enrolled in. Implementations of the module may be as simple as a quick questionnaire or survey exploring the patient's substance dependencies, social support systems, and other factors that may affect their motivation and adherence to plan requirements. Other more complex implementations may track the patients' motivation with time and change accordingly. Either way, the assessed motivation levels may be directly fed into the intelligent notification module 612 of the invention. This system uses patient activity tracking data to alert both the patient and their provider team of any notable lack of progress that may require additional attention and action. Patients' digital activity, indicated by system access and program input requirements, can be tracked by an implementation of this module and trigger on unique activity levels any set of appropriate notifications. These can be used for motivation and encouragement for the patient to continue making progress on their plans, as well as alerting providers when a patient has not reported to their enrolled and assigned programs with any activity. Depending on the patient's determined motivation assessment, these notifications may occur at unique intervals for each patient. For example, if a patient is assessed to have high motivational levels, infrequent reporting may be excused and therefore not trigger any notifications. Alternatively, a patient with potential adherence issues may be often notified of progress requirements, along with frequent notifications to their providers that progress on programs is not being made. The comprehensive reporting module 613 allows users to generate detailed reports and documents from the data stored and managed by the system. These reports can be exemplified but not limited to a list of programs and assets listed under a certain user or several users' available plans. Another instance of a potential reporting capability could be a comprehensive history of a patient's adherence and progress through an enrolled plan. These reports would provide users and professionals with the ability to track and manage the development of any aspect in patient care or asset and plan maintenance.

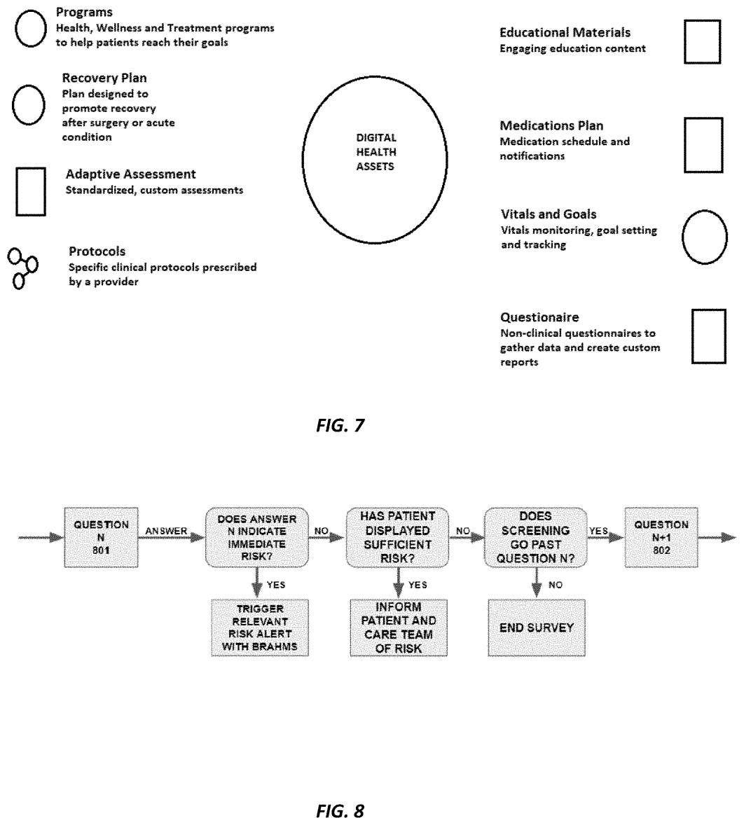

FIG. 7 illustrates several examples of digital assets that can be created and managed on the system, embodying the invention. The function of the assets ranges from assisting patients with recovery to notifying providers with a protocol of care for a specific set of patients. The technical makeup of each asset is vastly different from the other, and all they share in common is the ability to be created by a provider, and be assigned to other users of the platform. A general program may address any health or lifestyle goals a patient may have. A recovery plan may be more focused on steps that are aimed to help patients return to their normal lives from any operation, procedure or other significant lifestyle event. A questionnaire may be used to transfer important medical information from the patients to the providers. An adaptive assessment is an asset that may take patients' inputs and survey answers to make dynamic determinations of risk levels, medical needs, or any other medical assessment purpose. A protocol may be more useful for other providers rather than patients, and could represent an asset that gives professionals guidelines regarding best-practices for a wide variety of topics. Digital assets may also be implemented as a form of educational materials, not requiring any inputs or monitoring but simply provide patients or other providers with information relevant to their needs. A medication plan may require patients to confirm their medication intake regimen and provide patients with a schedule or notifications of missed medications. A vitals digital asset may represent the tracking of patients' physical attributes such as blood pressure or resting heart rate, and aggregate data that may be used for predictions or trend computations, ascertaining patients' future medical status.

FIG. 8 exemplifies a logic flow of an adaptive screening program. A provider may enroll a group of patients on the platform to complete the screening if necessary for medical purposes. The questionnaire may adaptively respond to a patient's answers in real time (a procedure embodied by a system referred to as BRAHMS). While accessing the digital assessment, a user's answer to a question 801 would be analyzed by the programmed logic. An example of this logic could be identifying whether a patient is at immediate risk of suicide. The system may detect such a risk using the patient's answers and alert the authorities or a relevant health provider in real time. The system may also use the patient's answers to detect risk in an intelligent way, and dynamically prompt users with relevant questions as they progress through the survey. Such an implementation is one example of a digital asset that providers could create and manage to help track and monitor their patients health and progress.

1. A computer implemented system which
a. Provides users a two-sided platform to browse and publish lifestyle, medical, and professional improvement plans.
b. Assesses the motivation levels of users and responds to their adherence to requirements accordingly.
c. Tiers users into motivation levels and informs relevant stakeholders when activity and progress are not reaching expected levels.
d. Stores information regarding users' progression in programs they are enrolled in.
e. Allows users to design and track the development of digital tools, referred to as assets, from a variety of templates.
f. Presents users with their live progress on their enrolled plans.
g. Users can enroll in joined programs with other users.

2. The method in which claim 1 is implemented, where digital assets can be implemented as, but not limited to, questionnaires, medical protocols, educational materials, and more.

3. The method in which claim 1 is implemented, where digital assets may require input from the patient, where inputs can be but are not limited to medical trackers such as heart rate or intake levels of a certain medication.

4. The method in which claim 1 is implemented, where providers would be able to create drafts of digital assets and store them in a private storage accessible only by the provider along with other private completed assets.

5. The method in which claim 1 is implemented, where providers would be able to publish digital assets publicly for all other platform users to view.

6. The method in which claim 1 is implemented, where providers would be able to enroll patients in digital assets, and patients would be able to access and view the requirements of such assets.

7. A computer-implemented method where access and notifications to outside parties are available, such as but not limited to direct alerts to local authorities.

8. The method in which claim 7 is implemented, where communication would be possible in reaction to patients' answers and input to surveys and other digital assets.

9. A computer-implemented system where dynamic and intelligent statistics-based assessment of medical conditions is used to survey and question patients for potential health risks.

10. A computer-implemented system which allows users to generate reports about data accessible to them, such as but not limited to ongoing progress under a subset of digital assets, a list of all patients under a provider's supervision, their specific adherence and overall health trends, as well as other comprehensive reports.
